(19) United States
(12) Patent Application Publication
(10) Pub. No.: US 2022/0243158 A1
(43) Pub. Date: Aug. 4, 2022

(54) PETRI DISH HOLDER AND LID OPENER

(71) Applicant: AMTIG ENGINEERING SOLUTIONS, LLC, Quincy, MI (US)

(72) Inventor: Steven GROHOLSKI, Quincy, MI (US)

(21) Appl. No.: 17/164,921

(22) Filed: Feb. 2, 2021

(51) Int. Cl. C12M 3/00; C12M 1/22

(52) U.S. Cl. CPC C12M 23/48; C12M 23/10

(57) ABSTRACT

A petri dish holder and lid opener comprising a petri dish lower portion holder including a lower shelf for holding the lower portion of a petri dish and a petri dish lid holder pivotally connected to the petri dish lower portion holder, with the petri dish lid holder having an upper shelf for holding a lid of a petri dish. The lower shelf has a bottom surface for holding the petri dish. The upper shelf has a bottom lip configured to be smaller than the lid of the petri dish. Rotation of the petri dish lid holder relative to the petri dish lower portion holder is configured to remove the lid of the petri dish from the lower portion of the petri dish.

### FIELD OF THE INVENTION

The present invention relates to petri dishes, and in particular to petri dish holder and lid opener.

### BACKGROUND OF THE INVENTION

Petri dishes are known for use in the medical and pharmaceutical industries as is well known to those skilled in the art. FIGS. 1A and lB illustrate a petri dish 10. The petri dish 10 includes a lower portion 12 and a lid 14. The lower portion 12 includes a circular bottom plate 16 and a cylindrical wall 18. The lid 14 includes a cylindrical skirt 20 that is marginally larger than the cylindrical wall 18 of the lower portion 12 and a top plate 22. The lid 14 is configured to be closely fit on the lower portion 12 as illustrated in FIG. lB. Previously, the lids 14 of the petri dishes 10 were removed from the lower portions 12 using hands or tongs.

### SUMMARY OF THE INVENTION

The present invention, according to one aspect, is directed to a petri dish holder and lid opener comprising a petri dish lower portion holder including a lower shelf for holding the lower portion of a petri dish and a petri dish lid holder pivotally connected to the petri dish lower portion holder, with the petri dish lid holder having an upper shelf for holding a lid of a petri dish. The lower shelf has a bottom surface for holding the petri dish. The upper shelf has a bottom lip configured to be smaller than the lid of the petri dish. Rotation of the petri dish lid holder relative to the petri dish lower portion holder is configured to remove the lid of the petri dish from the lower portion of the petri dish.

Another aspect of the present invention is to provide a petri dish assembly comprising a petri dish having a lower portion and a lid. The petri dish assembly also includes a petri dish holder and lid opener comprising a petri dish lower portion holder including a lower shelf holding the lower portion of the petri dish and a petri dish lid holder pivotally connected to the petri dish lower portion holder, with the petri dish lid holder having an upper shelf holding the lid of the petri dish. The lower shelf has a bottom surface supporting the petri dish. The upper shelf has a bottom lip smaller than the lid of the petri dish. Rotation of the petri dish lid holder relative to the petri dish lower portion holder removes the lid of the petri dish from the lower portion of the petri dish.

Yet another aspect of the present invention is to provide a method of opening a petri dish having a lower portion and a lid enclosing the lower portion. The method comprises providing a petri dish holder and lid opener comprising a petri dish lower portion holder including a lower shelf and a petri dish lid holder pivotally connected to the petri dish lower portion holder, with the petri dish lid holder having an upper shelf. The lower shelf has a bottom surface and the upper shelf having a bottom lip smaller than the lid of the petri dish. The method further includes inserting the petri dish into the petri dish holder and lid opener such that the lower portion of the petri dish is supported on the lower shelf and the lid is located above the bottom lip. The method also includes rotating the petri dish lower portion holder relative to the petri dish lid holder such that the lid of the petri dish is removed from the lower portion of the petri dish.

### BRIEF DESCRIPTION OF THE DRAWINGS

One or more embodiments of the present invention are illustrated by way of example and should not be construed as being limited to the specific embodiments depicted in the accompanying drawings, in which like reference numerals indicate similar elements.

FIG. 1A is a side view of a petri dish in a closed configuration.

FIG. 1B is a side view of the petri dish in an open configuration.

FIG. 2 is a perspective view of a petri dish holder and lid opener of the present invention.

FIG. 3 is a side view of the petri dish holder and lid opener of the present invention.

FIG. 4 is a front view of the petri dish holder and lid opener of the present invention.

FIG. 5 is a top view of the petri dish holder and lid opener of the present invention.

FIG. 6 is a perspective view of the petri dish holder and lid opener of the present invention holding the petri dish in the closed configuration.

FIG. 7 is a perspective view of the petri dish holder and lid opener of the present invention holding the petri dish in the open configuration.

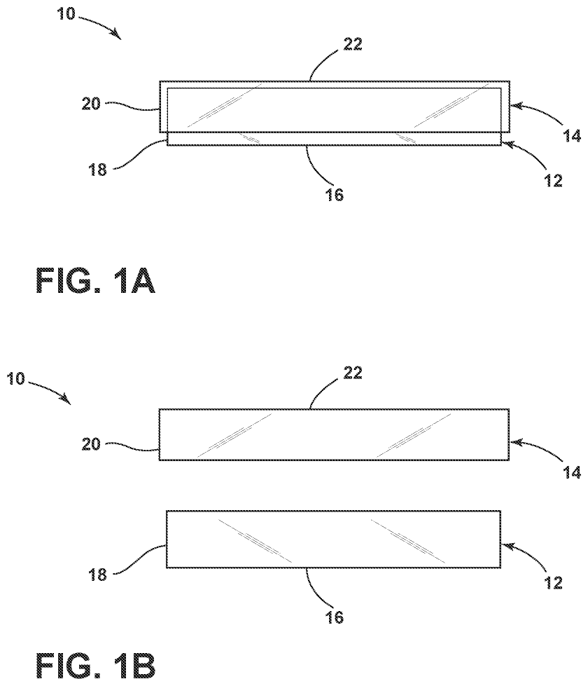

The specific devices and processes illustrated in the attached drawings and described in the following specification are simply exemplary embodiments of the inventive concepts. Hence, specific dimensions and other physical characteristics relating to the embodiments disclosed herein are not to be considered as limiting.

### DETAILED DESCRIPTION

For purposes of description herein, it is to be understood that the invention may assume various alternative orientations, except where expressly specified to the contrary. It is also to be understood that the specific devices and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

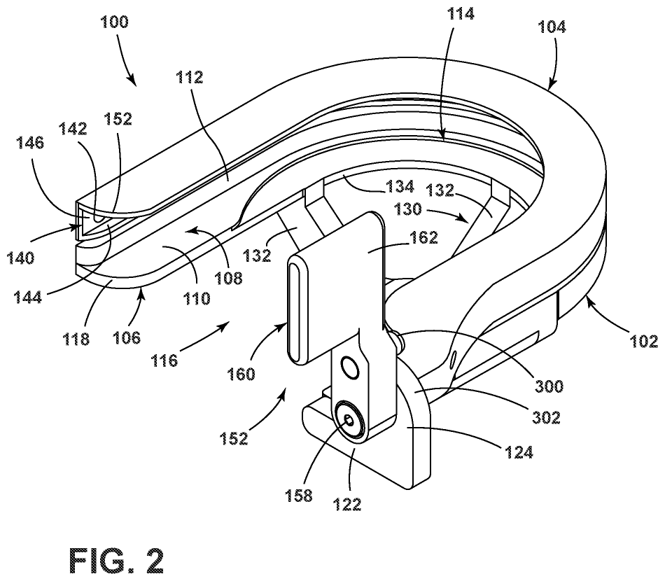

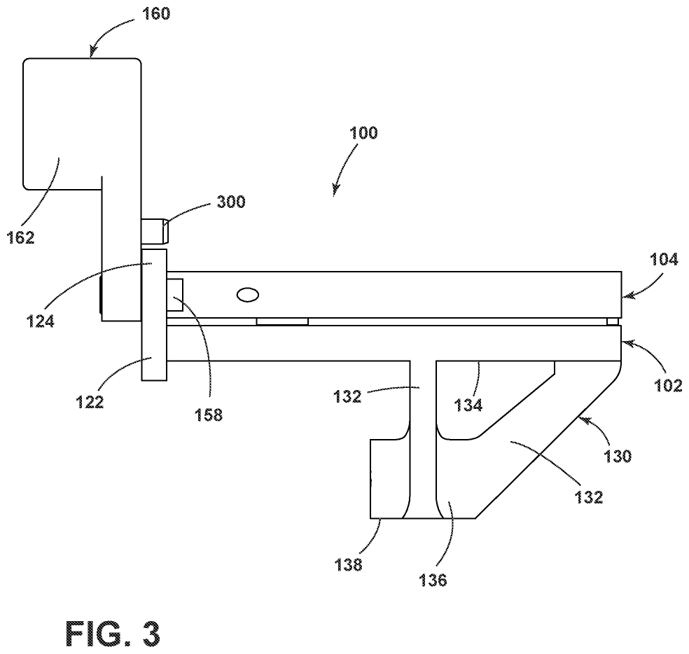

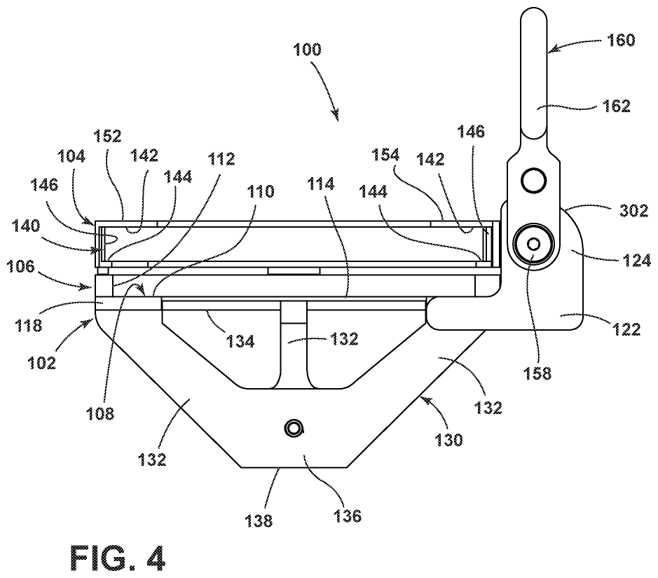

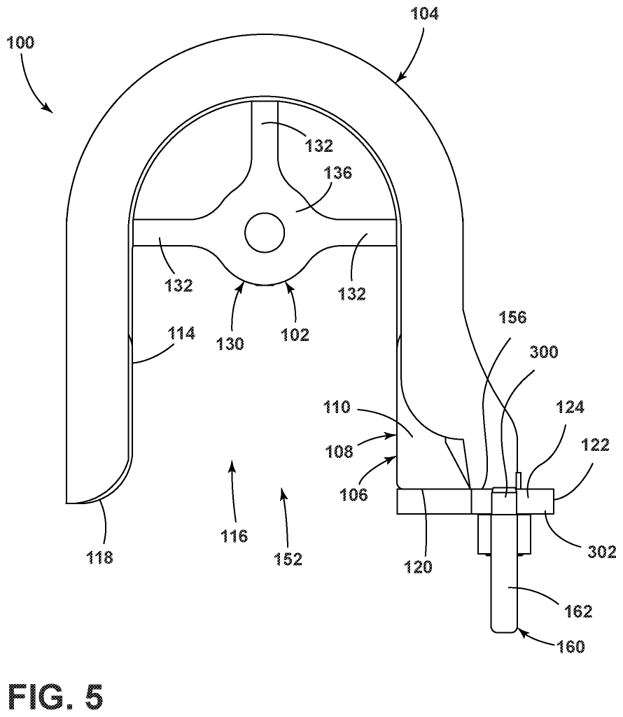

The reference number 100 (FIGS. 2-5) generally designates a petri dish holder and lid opener of the present invention. The petri dish holder and lid opener 100 allows a person to hold and open the petri dish 10, that is, remove the lid 14 from the lower portion 12, with a single hand. The petri dish holder and lid opener 100 of the present invention therefore allows for easy opening of the petri dish 10 and can provide a user thereof with a free hand for manipulating (e.g., removing) the material within the lower portion 12 of the petri dish 10. The petri dish holder and lid opener 100 includes a petri dish base holder 102 and a petri dish lid holder 104. The lid holder 104 rotates relative to the base holder 102 to remove the lid 14 of the petri dish 10 from the lower portion 12 of the petri dish 10.

In the illustrated example, the base holder 102 of the petri dish holder and lid opener 100 is configured to hold the lower portion 12 of the petri dish 10. The base holder 102 includes a U-shaped base receiver 106 configured to receive the lower portion 12 of the petri dish 10. The U-shaped base receiver 106 includes a U-shaped floor 108 having a top surface 110 and a U-shaped wall 112 extending upwardly from a periphery of the top surface 110. The U-shaped floor 108 includes a circular recess 114 having the same dimensions of the lower portion 12 of the petri dish 10. Therefore, the lower portion 12 of the petri dish 10 can be slid into the U-shaped base receiver 106 from an open mouth 116 thereof until the lower portion 12 of the petri dish 10 comes to rest within the circular recess 114.

The illustrated base holder 102 can be held by a user or fixed to a larger structure. As illustrated in FIGS. 2-5, the base holder 102 includes a leg assembly 130 for supporting the base holder 102. The leg assembly 130 includes a plurality of legs 132 extending downwardly and inwardly from a bottom surface 134 of the U-shaped floor 108 of the U-shaped base receiver 106. Each of the legs 132 converge and meet at a foot 136. The foot 136 includes a flat bottom surface 138 for supporting the base holder 102 and thereby the petri dish holder and lid opener 100 on the flat surface. It is also contemplated that the foot 136 can be connected to a support structure to support the petri dish holder and lid opener 100. Moreover, it is contemplated that the foot 136 and/or legs 132 can be held by a user of the petri dish holder and lid opener 100 during use of the petri dish holder and lid opener 100. It is contemplated that the foot 136 could include an opening 137 for accepting a fastener or pin therethrough for connecting the petri dish holder and lid opener 100 to a support.

In the illustrated example, the U-shaped floor 108 includes a first side front curved lip 118 at a first side of the open mouth 116 and a second side straight lip 120 at a second side of the open mouth 116. A mount plate 122 is connected to the front of the second side straight lip 120 and extends upwardly from the U-shaped floor 108. The mount plate 122 includes an upper portion 124 having a shaft opening 126 therethrough. A shaft of the lid holder 104 extends through the shaft opening 126, thereby connecting the lid holder 104 to the base holder 102 along with allowing the lid holder 104 to rotate relative to the base holder 102.

The illustrated lid holder 104 of the petri dish holder and lid opener 100 rotates relative to the base holder 102 to open the petri dish 10. The lid holder 104 includes an inwardly C-shaped recess 140 having a top U-shaped surface 142, a bottom U-shaped surface 144 and an outer U-shaped wall 146 extending between the periphery of the top U-shaped surface 142 and the bottom U-shaped surface 144. As shown in FIGS. 2-5, the top U-shaped surface 142 can be wider than the bottom U-shaped surface 144. The inwardly C-shaped recess 140 includes first side front curved lips 150 at a first side of an open mouth 152 thereof and a second side staggered lip 154 at a second side of the open mouth 152. The second side staggered lip 154 ends in a flat surface 156 that abuts against the mount plate 122 of the base holder 102. A shaft 158 extends from the second side staggered lip 154 and through the shaft opening 126 in the mount plate 122. On the other side of the mount plate 122 opposite the second side staggered lip 154, a flag-shaped lever 160 connected to the shaft 158 extends upward. The flag-shaped lever 160 includes a flange 162 extending away from the second side staggered lip 154 and parallel to the shaft 158.

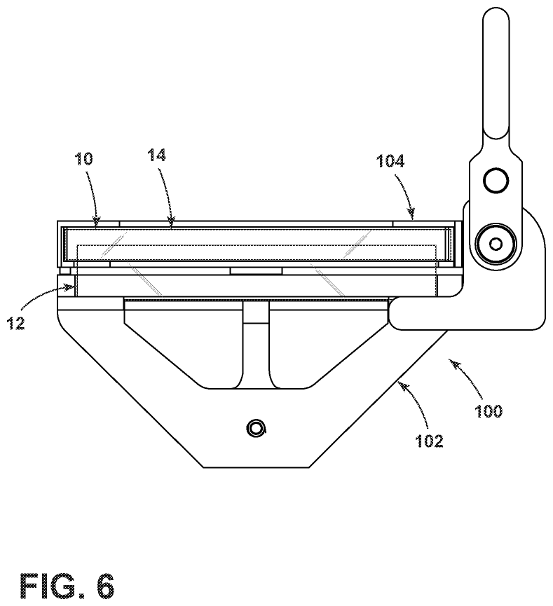

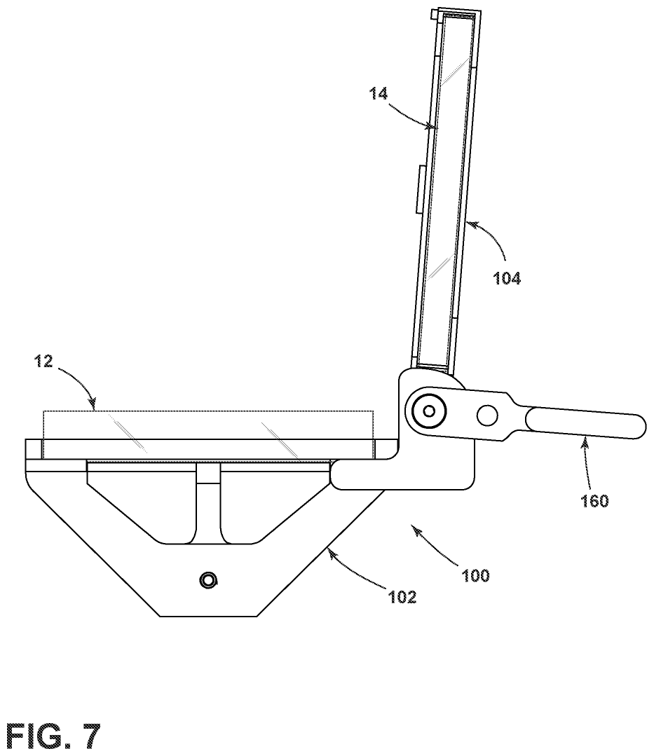

In the illustrated example, as the petri dish 10 is inserted into the petri dish holder and lid opener 100, the bottom portion 12 of the petri dish 10 will rest on the U-shaped floor 108 of the U-shaped base receiver 106. Simultaneously, the lid 14 of the petri dish 10 will be inserted into the inwardly C-shaped recess 140, with the cylindrical skirt 20 being adjacent the outer U-shaped wall 146 and between the top U-shaped surface 142 and the bottom U-shaped surface 144. FIG. 6 shows the petri dish 10 fully inserted into the petri dish holder and lid opener 100 while the petri dish holder and lid opener 100 and the petri dish 10 are in the closed position. As a user of the petri dish holder and lid opener 100 presses the flange 162 of the flag-shaped lever 160, the flag-shaped lever 160, the shaft 158 and the inwardly C-shaped recess 140 will rotate about the shaft opening 126 as illustrated in FIG. 7, thereby allowing access to the material 200 within the lower portion 12 of the petri dish 10. It is contemplated that the flag-shaped lever 160 can include a pin 300 extending parallel to the shaft 158 that runs over a curved top surface 302 of the mount plate 122 to help maintain proper alignment of the base holder 102 relative to the lid holder 104 as these two parts rotate relative to each other.

The petri dish holder and lid opener 100 of the present invention therefore allows for easy opening of the petri dish 10 and can provide a user thereof with a free hand for manipulating (e.g., removing) the material within the lower portion 12 of the petri dish 10. It is contemplated that the petri dish holder and lid opener 100 can be made of any material (e.g., plastic or metal). Moreover, it is contemplated that the petri dish holder and lid opener 100 can have a right hand configuration as shown which allows the petri dish holder and lid opener 100 to be easily manipulated with the right hand of the user or can have a left hand configuration which allows the petri dish holder and lid opener 100 to be easily manipulated with the left hand of the user, with the left hand configuration being a mirror image of the right hand configuration.

Although particular preferred embodiments of the invention have been disclosed in detail for illustrative purposes, it will be recognized that variations or modifications of the disclosed apparatus, including the rearrangement of parts, lie within the scope of the present invention.

What is claimed is:

1. A petri dish holder and lid opener comprising:
a petri dish lower portion holder including a lower shelf for holding a lower portion of a petri dish; and
a petri dish lid holder pivotally connected to the petri dish lower portion holder, the petri dish lid holder having an upper shelf for holding a lid of the petri dish;
the lower shelf having a bottom surface for holding the petri dish;
the upper shelf having a bottom lip configured to be smaller than the lid of the petri dish;
wherein rotation of the petri dish lid holder relative to the petri dish lower portion holder is configured to remove the lid of the petri dish from the lower portion of the petri dish.

2. The petri dish holder and lid opener of claim 1, wherein:
the upper shelf is arcuate and has a C-shaped cross-section which opens inwardly.

3. The petri dish holder and lid opener of claim 1, wherein:
the petri dish lower portion holder has a plurality of downwardly depending legs extending downwardly from the lower shelf.

4. The petri dish holder and lid opener of claim 3, wherein:
the plurality of downwardly depending legs converge at a foot.

5. The petri dish holder and lid opener of claim 1, wherein:
the petri dish lower portion holder has a slot; and
the petri dish lid holder has a shaft extending through the slot, the shaft and the slot forming a pivotal connection between the petri dish lower portion holder and the petri dish lid holder.

6. The petri dish holder and lid opener of claim 5, wherein:
the petri dish lid holder includes a lever connected to the shaft.

7. The petri dish holder and lid opener of claim 1, wherein:
the lower shelf includes a circular recess configured to closely conform to an exterior shape of the lower portion of the petri dish.

8. A petri dish assembly comprising:
a petri dish having a lower portion and a lid; and
a petri dish holder and lid opener comprising a petri dish lower portion holder including a lower shelf holding the lower portion of the petri dish and a petri dish lid holder pivotally connected to the petri dish lower portion holder, the petri dish lid holder having an upper shelf holding the lid of the petri dish;
the lower shelf having a bottom surface supporting the petri dish;
the upper shelf having a bottom lip smaller than the lid of the petri dish;
wherein rotation of the petri dish lid holder relative to the petri dish lower portion holder removes the lid of the petri dish from the lower portion of the petri dish.

9. The petri dish assembly of claim 8, wherein:
the upper shelf is arcuate and has a C-shaped cross-section which opens inwardly.

10. The petri dish assembly of claim 8, wherein:
the petri dish lower portion holder has a plurality of downwardly depending legs extending downwardly from the lower shelf.

11. The petri dish assembly of claim 10, wherein:
the plurality of downwardly depending legs converge at a foot.

12. The petri dish assembly of claim 8, wherein:
the petri dish lower portion holder has a slot; and
the petri dish lid holder has a shaft extending through the slot, the shaft and the slot forming a pivotal connection between the petri dish lower portion holder and the petri dish lid holder.

13. The petri dish assembly of claim 12, wherein:
the petri dish lid holder includes a lever connected to the shaft.

14. The petri dish assembly of claim 8, wherein:
the lower shelf includes a circular recess configured to closely conform to an exterior shape of the lower portion of the petri dish.

15. A method of opening a petri dish having a lower portion and a lid enclosing the lower portion, the method comprising:
providing a petri dish holder and lid opener comprising a petri dish lower portion holder including a lower shelf and a petri dish lid holder pivotally connected to the petri dish lower portion holder, the petri dish lid holder having an upper shelf, the lower shelf having a bottom surface and the upper shelf having a bottom lip smaller than the lid of the petri dish;
inserting the petri dish into the petri dish holder and lid opener such that the lower portion of the petri dish is supported on the lower shelf and the lid is located above the bottom lip; and
rotating the petri dish lower portion holder relative to the petri dish lid holder such that the lid of the petri dish is removed from the lower portion of the petri dish.
